(19) United States
(12) Patent Application Publication
(10) Pub. No.: US 2006/0241204 A1
(43) Pub. Date: Oct. 26, 2006

(54) Use of butylene terephthalate for producing thermoplastic dental prostheses

(75) Inventor: Karl-Heinz Renz, Jettingen (DE)

Correspondence Address: Norris, McLaughlin & Marcus P.A., 18th Floor, 875 Third Avenue, New York, NY, 10022, US

(73) Assignee: Heraeus Kulzer GmbH, Hanau (DE)

(21) Appl. No.: 11/398,416

(22) Filed: Apr. 5, 2006

(30) Foreign Application Priority Data
Apr. 20, 2005 (DE) 10 2005 018 450.2

(51) Int. Cl. A61K 6/10

(52) U.S. Cl. 523/109

(57) ABSTRACT

The use of cyclic oligomeric butylene terephthalate (CBT™) for producing thermoplastic dental prostheses, or for producing starting materials for thermoplastic dental prostheses are described, in addition to starting materials containing CBT™ for producing thermoplastic dental prostheses, and a method for producing thermoplastic dental prostheses.

The present invention relates to the use of butylene terephthalate for producing thermoplastic dental prostheses, in particular dentures, artificial teeth, crowns, or bridges, for producing starting materials for thermoplastic dental prostheses, the starting materials, and a method for producing thermoplastic dental prostheses using butylene terephthalate.

### BACKGROUND OF THE INVENTION

A distinction is made between three main material classes for producing complete prosthetic appliances: dual-component materials based on polymethyl methacrylate (PMMA); PMMA-free, heat-curing materials; and injected-molded compounds that can be thermoplastically processed.

Thermoplastic dental prostheses may be produced in the injection molding process from polyacetals or mixtures of PMMA/polyamide. Disadvantages are the very high processing temperature and difficult process conditions, such as high material viscosity and the very high processing pressures which result. For example, interdental spaces are not easily filled.

Cyclic oligomers of butylene terephthalate have been known for quite some time (for example, Mueller, F. J. et al., “Synthesis of Cyclic Oligomers of Butylene Terephthalate,” Makromol. Chem. 184, 2487-95 (1983)).

Cyclics Corporation, Schenectady, N.Y., USA, markets cyclic oligomeric butylene terephthalate (CBT™), produced by using commercially available polybutylene terephthalate (PBT). CBT™ resins have a relatively low melting point, are very flowable, and result in a cured synthetic material with good mechanical properties which can be molded into complex shapes. Furthermore, in contrast to traditional epoxy resins the material can be easily recycled. The material and its production are described in U.S. Pat. No. 6,420,048 B1, U.S. Pat. No. 6,420,047 B1, and U.S. Pat. No. 6,369,157 B1, for example.

When CBT™ is mixed with initiators, a reverse reaction occurs which results in PBT. CBT™ having the advantage that it has a much lower viscosity than PBT, and thus is better able to penetrate fine channels. Use may be made of this feature with surprisingly good results in the production of dental prostheses. Thus, for example, dental interstices may be filled with a practically 100% thermoplastic material. In this manner, compensation is made for the disadvantages of thermoplastic dental prosthesis materials previously encountered.

### SUMMARY OF THE INVENTION

The present invention therefore relates to the
use of cyclic oligomeric butylene terephthalate for producing thermoplastic dental prostheses, or for producing starting materials for thermoplastic dental prostheses,
   in particular for producing dentures (also fiber-reinforced), artificial teeth, crowns, or bridges (also fiber-reinforced), and starting materials therefor.

The subject matter of the invention represents a favorable alternative to the processing of thermoplastic dental prosthesis materials in the “thin liquid” state, and thus essentially at high temperatures. The above-mentioned disadvantages of working with thermoplastic materials are largely avoided.

### DETAILED DESCRIPTION

The invention further relates to a starting material for producing thermoplastic dental prostheses, containing

| | | |
|---|---|---|
| CBT ™ | 50-99.9% | by weight |
| Polycaprolactone | 0-20% | by weight |
| Initiator components | 0.001-2% | by weight |
| Inorganic fillers | 0-50% | by weight |
| Organic fillers | 0-50% | by weight |

The starting material may also contain additional common additives, such as dyes or pigments. Organotin or titanate compounds are primarily used as initiators.

Material-related advantages compared to conventional thermoplastic materials used in dentistry are a very balanced ability to adjust the rigidity and impact strength. Thus, relatively rigid molded bodies with high impact strength may be produced.

The invention further relates to a method for producing thermoplastic dental prostheses, in particular dentures, by the following steps:
Modeling of the part(s) to be replaced in wax;
Embedding the wax model;
Curing the embedded material;
Removal of the wax;
Introduction of a liquid CBT™ mixture into the resulting cavity;
Curing the CBT™ mixture.
   Correspondingly, teeth according to the conventional procedure, for example, are placed in wax, then after the teeth are surrounded with an embedding material the wax is removed, and the resulting cavity is filled with a liquid CBT™ mixture which is then cured.

### EXAMPLE

A composition containing

| CBT ™ | 99.7% by weight |
|---|---|
| Corresponding initiator components | 0.3% |

is poured into plates at approximately 170° C. and cured at approximately 200° C.

This results in PBT having the following properties:

Flexural strength (ISO 1567): 85 MPa

Flexural modulus (ISO 1567): 2500 MPa

Impact strength (ISO 1567, Amendment 1): 1.5 MPa

1. A method for producing thermoplastic dental prostheses or starting materials for thermoplastic dental prostheses, which comprises producing said thermoplastic dental prostheses or starting materials for thermoplastic dental prostheses with cyclic oligomeric butylene terephthalate.

2. Method according to claim 1, wherein said thermoplastic dental prosthesis are dentures, artificial teeth, crowns, or bridges.

3. Starting material for producing thermoplastic dental prostheses, comprising

| | | |
|---|---|---|
| Cyclic oligomeric butylene terephthalate | 50-99.9% | by weight |
| Polycaprolactone | 0-20% | by weight |
| Initiator components | 0.001-2% | by weight |
| Inorganic fillers | 0-50% | by weight |
| Organic fillers | 0-50% | by weight |

4. Method for producing thermoplastic dental prostheses by the following steps:
Modeling of the part(s) to be replaced in wax;
Embedding the wax model;
Curing the embedded material;
Removal of the wax;
Introduction of a starting material according to claim 3, in liquid form, into the resulting cavity;
Curing the starting material.

5. Method of claim 4, wherein said dental prostheses are dentures.
